SEQUENCE LISTING (4 sequences)

SEQ ID NO: 1 - DNA, 1377 bp, Arabidopsis thaliana L. Heynh.
atgcccatgttaatggcgacacgtatcgatataatccaaaagcttaatgtatatccaaggtttcaaaaccatgacaagaagaaactaatcactctctccaatttggaccgtcagtgtcctttactcatgtactctgtcttcttctacaagaataccacaactcgtgactttgactccgtcttctccaacctgaagctcgggctggaggagactatgtctgtgtggtatcccgcggcagggagactgggtttggacggaggtggctgcaagctcaacatccggtgtaacgatggtggcgcagtcatggtggaggcggtggcgacaggtgtcaagttgtccgagcttggtgatttgactcagtacaatgagttttatgagaatttagtttacaagccttccttggatggtgatttctctgtgatgcctcttgttgttgctcaggtgacaagatttgcatgtggaggttactcaattggaatcggtacaagccactctctatttgatggaatctcagcttacgaattcattcacgcgtgggcctccaactctcacattcacaacaaatccaacagcaagattactaataaaaaggaagatgtggtcatcaaaccggttcatgatcgacgaaatctactggttaaccgggatgctgtccgagaaaccaatgctgcagccatttgtcatctgtaccagttgatcaaacaggcgatgatgacctatcaggagcaaaaccgtaacttagagttaccagactctggttttgtgatcaaaacgttcgagcttaatggcgatgcgatagaaagcatgaagaagaaatcactagaagggttcatgtgctcctcctttgagtttcttgctgctcatttgtggaaggcaagaacaagggctttagggttgaggagagacgccatggtgtgtttacaattcgcagtggacataaggaaaagaacggagacaccgctgccagaagggttttccggcaacgcatacgtgcttgcctcggtggcatccaccgccagagaattacttgaagagctaacactcgagtcaatagtcaacaagatcagagaagccaagaaatcaattgaccaaggttacataaactcttacatggaagcactcggaggtagtaatgacggaaatctccctcctctcaaagagctaaccctaatctccgactggacaaaaatgccatttcacaatgttggctttggcaacggcggcgagccagcggattacatggccccactgtgtccaccggtgccacaagttgcttatttcatgaagaaccctaaagatgccaaaggtgttcttgtgaggattggcttggacccacgagatgttaatggcttttcaaatcatttccttgattgctaa

SEQ ID NO: 2 - protein, 458 aa, Arabidopsis thaliana L. Heynh.
MPMLMATRIDIIQKLNVYPRFQNHDKKKLITLSNLDRQCPLLMYSVFFYKNTTTRDFDSVFSNLKLGLEETMSVWYPAAGRLGLDGGGCKLNIRCNDGGAVMVEAVATGVKLSELGDLTQYNEFYENLVYKPSLDGDFSVMPLVVAQVTRFACGGYSIGIGTSHSLFDGISAYEFIHAWASNSHIHNKSNSKITNKKEDVVIKPVHDRRNLLVNRDAVRETNAAAICHLYQLIKQAMMTYQEQNRNLELPDSGFVIKTFELNGDAIESMKKKSLEGFMCSSFEFLAAHLWKARTRALGLRRDAMVCLQFAVDIRKRTETPLPEGFSGNAYVLASVASTARELLEELTLESIVNKIREAKKSIDQGYINSYMEALGGSNDGNLPPLKELTLISDWTKMPFHNVGFGNGGEPADYMAPLCPPVPQVAYFMKNPKDAKGVLVRIGLDPRDVNGFSNHFLDC

SEQ ID NO: 3 - DNA, 24 bp, Artificial Sequence
  GS4
acattctacaactacatctagagg

SEQ ID NO: 4 - DNA, 17 bp, Artificial Sequence
  GS6
cggccgccccggggatc

(19) United States
(12) Patent Application Publication
(10) Pub. No.: US 2015/0299721 A1
(43) Pub. Date: Oct. 22, 2015

(54) BIOMASS PRODUCTION INCREASING GENE AND TRANSGENIC PLANT USING SAME

(71) Applicant: POSTECH ACADEMY-INDUSTRY FOUNDATION, Pohang-si, Gyeongsangbuk-do (KR)

(72) Inventors: Ildoo HWANG, Pohang-si, Gyeongsangbuk-do (KR); Sunhwa CHOI, Seoul (KR)

(73) Assignee: POSTECH ACADEMY-INDUSTRY FOUNDATION, Pohang-si, Gyeongsangbuk-do (KR)

(21) Appl. No.: 14/440,888

(22) PCT Filed: Nov. 7, 2013
(86) PCT No.: PCT/KR2013/010090

§ 371 Date: May 6, 2015

(30) Foreign Application Priority Data
Nov. 7, 2012 (KR) 10-2012-0125652

(51) Int. Cl. C12N 15/82; C12N 9/10

(52) U.S. Cl. CPC C12N 15/8255; C12N 9/1025

(57) ABSTRACT

The present invention relates to, inter alia, a gene which increases biomass production isolated from Arabidopsis thaliana, and a method for producing a transgenic plant by using same. More specifically, the present invention provides, inter alia, a composition, a recombinant expression vector and a transgenic plant for increasing plant biomass production, comprising a base sequence coding for the amino acid sequence of sequence number 2. Consequently, by using the gene for increasing biomass production of the present invention, it is possible to obtain a transgenic plant with which the amount of biomass production is increased and, ultimately, it is expected that same can be used in order to increase starting materials for the pulp and papermaking industries, and starting materials for bioethanol.

### TECHNICAL FIELD

The present invention relates to a biomass production increasing gene and a method of producing a transgenic plant using the same.

### BACKGROUND ART

Due to high oil prices, plant biomasses are being significantly considered as raw materials for generating bioenergy. First generation biofuel production using saccharification of starch in grains caused an increase in crop prices and food and an economic crisis. Therefore, no related technology has been developed recently. On the other hand, research on second generation bioenergy using lignocellulose in non-food biomass crops such as silver grass, switchgrass, and poplars is being actively conducted. However, in order to substantially produce a great amount of energy in place of fossil fuels, efforts of increasing and continuously keeping an amount of plant biomass production are generally necessary. For this purpose, research on a cambium greatly influencing secondary growth, vascular tissue development, and a growth regulatory mechanism is necessary.

Vascular tissues of plants serve as a transport pathway for substances such as water and nutrients necessary for plant growth and development and form a distinctive structure in which cells having specific functions are collected. In primary growth, that is, length growth, vascular tissues in a stem are generated by the uppermost apical meristem, and include meristems such as a procambium, a primary phloem, and a xylem. Enlarged cambium cells are formed in vascular tissues between bundles for secondary growth over time. Using the cambium cells as initial cells, a secondary phloem and xylem are developed in different directions and thus a closed circular structure is formed.

Various plant growth hormones are involved in development of vascular tissues in the stem. Auxin and cytokinin, which are closely related to a general activity of the meristem, influence cambium cell division. Gibberellin and ethylene are involved in a cambium activity. Also, a brassinosteroid regulates the number of vascular bundles or a pattern thereof, which is determined by maxima that are generated by regulating movement of auxin. It has been known that specific genes are involved in tissue-specific formation in addition to the above examples. For example, all of ATHB8, CNA (ATHB15), PHB, PHV, and REV included in an HD-ZIP III gene group are generally expressed in the cambium of vascular tissues. It has been known that development directions of the phloem and the xylem and differentiation of the xylem in each vascular bundle are regulated by these genes. Also, another regulating factor KANADI regulates expression of the HD-ZIP III described above through miRNA165/166 and has been reported to regulate vascular tissue formation according to an interaction with auxin.

Although such a complex inter-regulation mechanism has been relatively well studied, a case in which an amount of biomass that can be actually produced is changed through a modification of related genes has not been reported up to now. This means that there is a limit to increasing an amount of biomass production using a factor involved in specific organization development.

Meanwhile, Arabidopsis thaliana has a short first generation period of about 6 weeks from germination until the next seed forms, and when a chemical substance is used, various forms of mutants can be easily produced. Also, it can be easily grown in a glass container since a size thereof is small, and a genome size is small. Due to these advantages, Arabidopsis thaliana is used as a model plant for plant research in many cases. Arabidopsis thaliana has a height of about 30 cm, forms a flower bud in about 3 weeks after germination under a long-day condition, may obtain an initial seed in 5 to 6 weeks, and may perform self-pollination and artificial hybridization. The most significant feature thereof is the smallest genome size, 1×10⁸ base pairs/monoploid, among known phanerogamous plants. The number of chromosomes is 2n=10, and a repeat sequence is small. Howard Goodman and others (MIT USA) completed a genetic map labeled with a restriction enzyme fragment length polymorphism (RELP), and the National Science Foundation (NSF)-funded “Arabidopsis thaliana” genome project was conducted from 1990.

The inventors used Arabidopsis thaliana having the above advantages and studied to discover a new biomass production increasing gene.

### DISCLOSURE

### Technical Problem

The inventors discovered a gene that increases a cambium activity of a plant in order to increase an amount of biomass production of the plant serving as a raw material of bioenergy, produced a transgenic plant in which an amount of biomass production increases using the discovery, and completed the present invention based thereon.

Therefore, the present invention provides a composition of increasing biomass production of a plant including base sequences encoding amino acid sequences of SEQ ID NO: 2, and a plant transformed by the composition.

The present invention also provides a method including an operation of transforming a plant using the composition.

However, the scope of the present invention is not limited to the above-described objects, and other unmentioned objects may be clearly understood by those skilled in the art from the following descriptions

### Technical Solution

In order to address the above problems, in the present invention, cross-sections of stems were observed using a FOX hunting system (Full-length cDNA Over-eXpressing gene hunting system) in which about 12,000 Arabidopsis thaliana full-length cDNAs are over-expressed, plants having an altered cambium activity were selected, and overexpressed genes that are expected to influence a phenotype were isolated and identified. Also, the experiment proved that these genes actually induced phenotypes, and various biomass-related phenotypes expressed in corresponding gene over-expressomes were identified.

Accordingly, the present invention provides a composition of increasing biomass production of a plant including base sequences encoding amino acid sequences of SEQ ID NO: 2.

According to an aspect of the present invention, the base sequences include base sequences of SEQ ID NO: 1.

The present invention also provides a composition of increasing biomass production including a plant expression recombinant vector into which base sequences encoding amino acid sequences of SEQ ID NO: 2 are inserted.

The present invention also provides a plant that is transformed using the composition and the plant has increased biomass production.

The present invention also provides a method of increasing biomass production of a plant, including transforming a plant using the composition.

### Advantageous Effects

When a biomass production increasing gene of the present invention, that is, a cambium activity regulating gene BAT1 (at4g31910) is used, it is possible to produce a transgenic plant having an increased cambium activity. Therefore, according to the present invention, it is possible to supplement raw materials of pulp and paper industries ultimately, and ensure raw materials of a bioethanol according to an increase in an amount of lignocellulose. In addition, the present invention can be expected to be used as heating and electricity production materials in the form of firewood, pellet or the like. Also, it is possible to prevent lodging due to a weight of grains by enhancing a physical supporting force of a stem when a grain variety having an increased amount of outflow water is produced.

### DESCRIPTION OF DRAWINGS

FIG. 1A shows a phenotype in which the number of vascular tissues of a BAT1 gene-overexpressing plant decreased more than that of wild type, FIGS. 1B and 1C shows a dwarf phenotype similar to bri1-1 that is a mutant defective in signal transmission of a brassinosteroid, and FIG. 1D shows an expression pattern of brassinosteroid signal transmission marker genes supporting such a phenotype.

FIG. 2A shows selection of a BAT1 gene-deficient T-DNA insertion mutant and a selected bat1-1-deficient mutant, FIGS. 2B and 2C show various phenotypes of bat1-1-deficient mutants in which biomass increased more than that of a wild type and show bat1-1-deficient mutants having a phenotype in which a length of a stem is greater than that of the wild type, and FIG. 2D shows the increased number of vascular tissues.

FIG. 3 shows a position in which BAT1 is expressed in a plant. BAT1 is expressed in a nucleus and an endoplasmic reticulum in a cell (A), is expressed in vascular tissues of a leaf, a cotyledon and a root in a juvenile period (B and C), and is expressed in vascular tissues of a stem in an adult period (D).

FIG. 4 shows the measured amount of intermediate metabolites of each brassinosteroid that is decreased in a plant due to overexpression of BAT1 genes.

FIG. 5 shows the observation results of various phenotypes according to BAT1 overexpression that are restored to phenotypes similar to those of the wild type by treating with an internal or external brassinosteroid. In FIG. 5A, a hypocotyl height decreased due to BAT1 gene overexpression becomes similar to a length of the wild type by treating with a brassinosteroid. In FIG. 5B, it was observed that a dwarf phenotype of an adult was also restored by treating with a brassinosteroid. In FIG. 5C, when a BAT1 gene-overexpressing plant is genetically cross fertilized with a DWF4 gene-overexpressing plant that is a mutant in which a brassinosteroid is excessively accumulated, a phenotype thereof becomes similar to that of the wild type.

FIG. 6A shows base sequences of BAT1 (at4g31910) and FIG. 6B shows amino acid sequences.

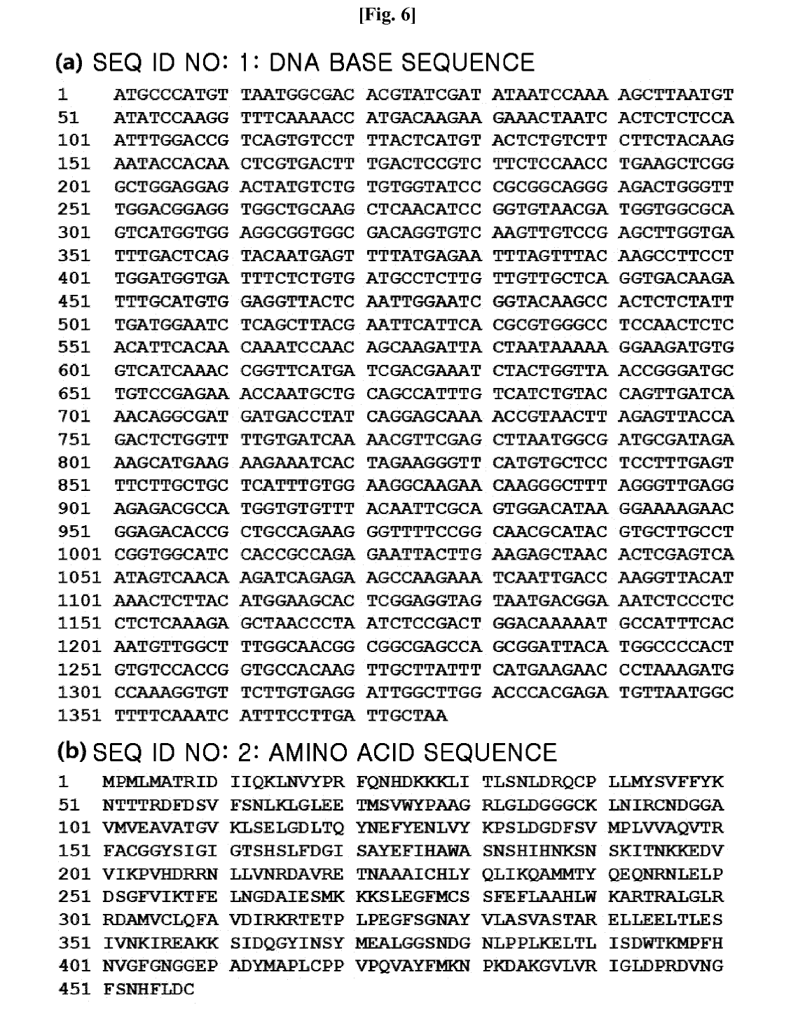

### MODES OF THE INVENTION

As described above, in order to study a method of increasing biomass production, the inventors observed morphological variations in vascular tissues in a stem resulting from use of Arabidopsis thaliana full-length cDNA and features of primary and secondary growths shown on an outer side of the stem.

As a result, the inventors verified that BAT1 (at4g31910), which is a gene over-expressed in a transgenic plant in which the number of cells of a cambium increased, may influence a biomass increase and metabolism of a brassinosteroid, and completed the present invention.

Therefore, the present invention provides a composition of increasing biomass production of a plant including base sequences encoding amino acid sequences of SEQ ID NO: 2.

Also, the base sequences encoding amino acid sequences of SEQ ID NO: 2 include base sequences of SEQ ID NO: 1.

Meanwhile, due to degeneracy of a codon, a variation in base sequences may not cause a change in proteins. Therefore, it is apparent to those skilled in the art that base sequences used in the present invention are not limited to the base sequences of SEQ ID NO: 1 described in the accompanying sequence list.

Also, in order to obtain an effect of increasing an amount of biomass production, a plant expression recombinant vector may be used to introduce genes. Therefore, the present invention provides a composition of increasing biomass production including a plan expression recombinant vector into which the base sequences encoding amino acid sequences of SEQ ID NO: 2 are inserted. The plant expression recombinant vector used in the present invention may include pCB302ES, pCXSN, pINDEX3, pBI121, or pgR106, but the present invention is not limited thereto.

Also, the present invention provides a plant transformed by a composition among compositions of the present invention. The plant used in the present invention may include Arabidopsis thaliana, tobacco, tomato, silver grass, switchgrass, or a poplar, and preferably Arabidopsis thaliana, but the present invention is not limited thereto.

Also, the present invention provides a method of increasing biomass production of a plant, and the method including an operation of transforming a plant using one of the compositions of the present invention.

In examples of the present invention, a FOX hunting system (Full-length cDNA Over-eXpressing gene hunting system) in which about 12,000 Arabidopsis thaliana full-length cDNAs are over-expressed was used to observe morphological variations in vascular tissues in a stem and features of primary and secondary growths shown on an outer side of the stem.

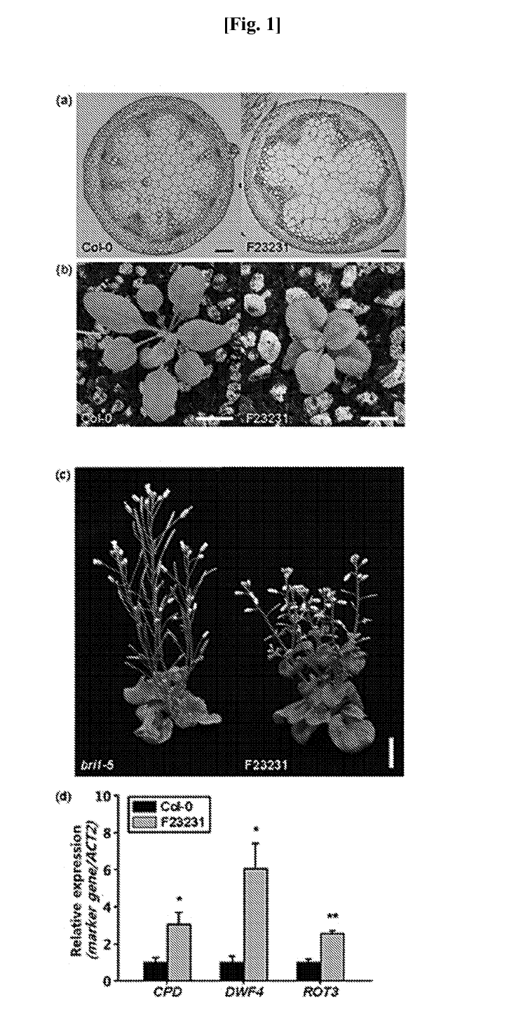

As a result, it can be observed that, in a F2323 FOX hunting system transformant among them, the number of cells of a cambium that is a phenotype directly related to a biomass increase is changed. It was determined that the selected transgenic plant F23231 showed a phenotype in which the numbers of cells and layers of a procambium are changed and the cambium and the number of vascular tissues decreased more than those of a wild type (refer to FIG. 1A). In addition, it can be observed that a surface area of a rosette leaf and a length of a petiole significantly decreased more than those of the wild type (refer to FIG. 1B), and a length of a stem of an adult is only 70% that of the wild type (refer to FIG. 1C). It can be expected from the results that a biomass increase and decrease may be influenced by manipulating the selected gene and regulating its function. It has been well known that development of such vascular tissues and a defect phenotype of an overall development process are regulated by various environmental conditions and hormone signal transmission. In particular, it can be understood that the above selected F23231 plant has a phenotype similar to that of a mutant defective in signal transmission of a brassinosteroid (BR), which is a steroid hormone of a plant. A bri1-5 mutant compared in FIG. 1C is a mutant defective in BRI1 known as a membrane receptor of a brassinosteroid, and was used as a representative mutant defective in brassinosteroid signal transmission.

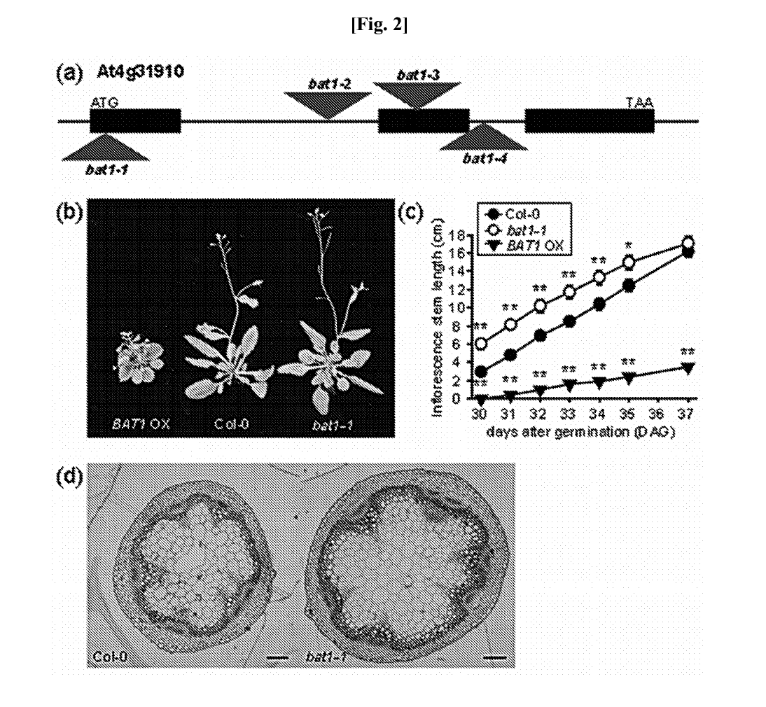

Therefore, in order to find a gene mediating the phenotype, an experiment in which over-expressed genes were isolated and identified from the selected transformant F23231 was performed. As a result, it was determined that the selected transformant includes over-expressed at4g31910 genes having an acylatransferase domain and named BAT1 (BR-related AcylTransferase 1) in association with a function of a protein later. In order to know a function of the gene, BAT1-deficient mutants (knock out mutants of genes through T-DNA insertion) were obtained (FIG. 2A), and a bat1-1-deficient mutant among them was used for an experiment. Unlike the BAT1-overexpressing plant, it was observed that the bat1-1-deficient mutant had a length of a stem that increased more than that of the wild type (refer to FIGS. 2B and 2C), and development of vascular bundles and a thickness of the stem significantly increased more than those of the wild type (refer to FIG. 2D). This proved that biomass directly increased by manipulating BAT1 genes.

Also, the inventors attempted to prove a hypothesis that the gene is directly related to an increase in biomass using a BAT1 gene-overexpressing or BAT1 gene-deficient mutant phenotype, and this gene may also influence metabolism of a brassinosteroid. It has been known that a brassinosteroid hormone used in a cell needs a deactivation mechanism for deactivating later or converting it into a storing form in the cell. In addition, an acylated form of a brassinosteroid was identified in several plant species, but research on related enzymes has not been well reported.

The above identified BAT1 includes an acyltransferase domain and shows a phenotype similar to that of the mutant defective in brassinosteroid signal transmission when BAT1 genes are over-expressed. Accordingly, the newly identified BAT1 can be expected as an enzyme related to deactivation of a brassinosteroid.

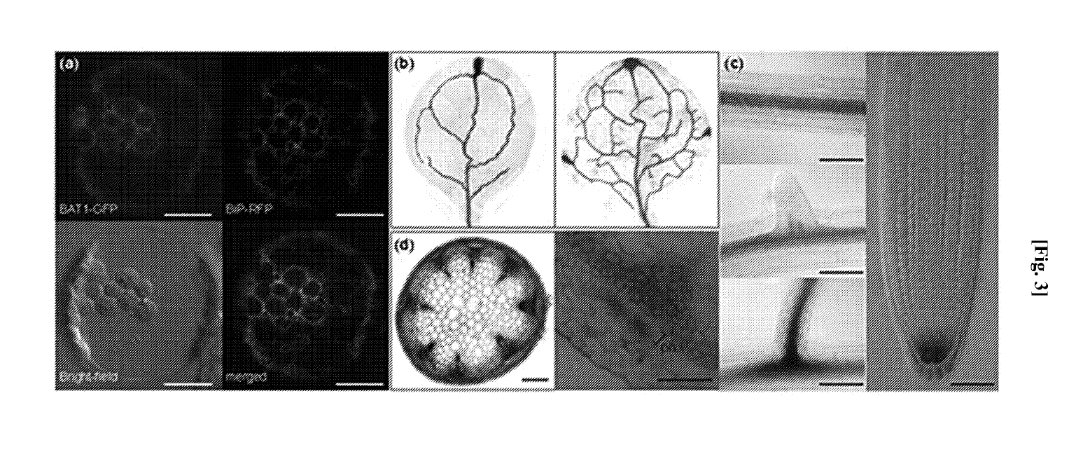

Also, in order to identify a molecular mechanism and a function of the BAT1 gene, an expression position of a BAT1 protein was observed in the cell and for each tissue according to a development time (refer to FIG. 3). In the cell, BAT1 is expressed in the nucleus and the endoplasmic reticulum (refer to FIG. 3A). The endoplasmic reticulum in which BAT1 is expressed is a position in which a well-known gene related to biosynthesis of the brassinosteroid hormone is provided. Accordingly, it can be expected that biosynthesis and metabolism of a brassinosteroid are efficiently performed in the same position in the cell. In addition, in a development stage in a juvenile period, BAT1 is expressed in vascular tissues of a leaf and a cotyledon (refer to FIG. 3B), and a root (refer to FIG. 3C). In a development stage in an adult period, it was observed that BAT1 was expressed in a stem, and particularly, expressed specifically to vascular tissues (refer to FIG. 3D). Such a tissue-specific BAT1 expression can be evidence that shows a close relation with a biomass increase and decrease.

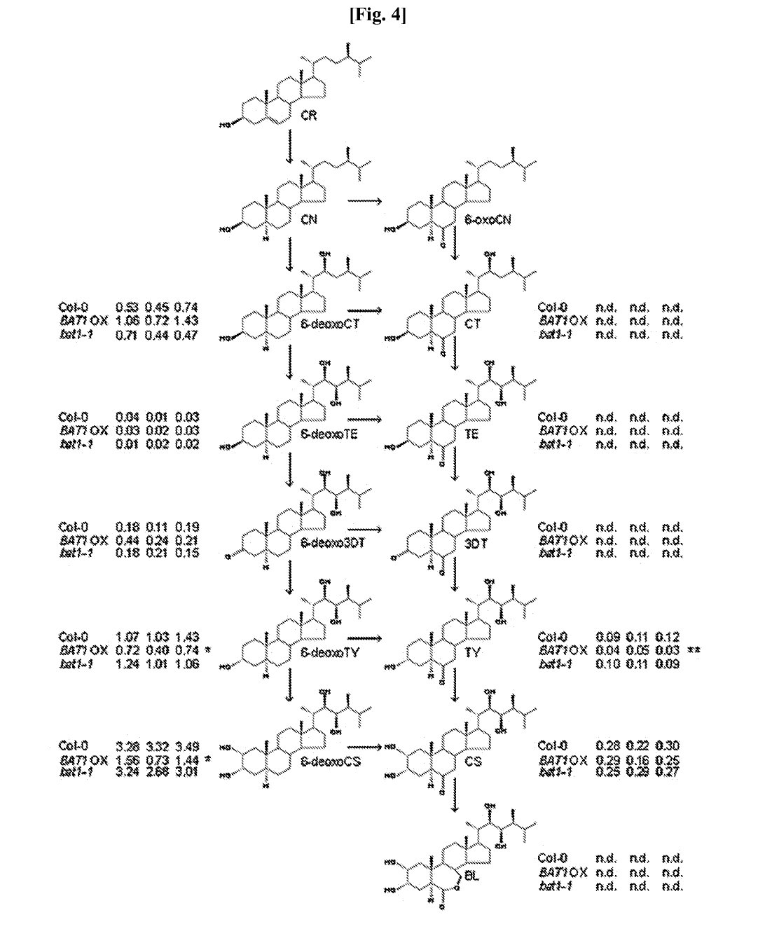

Also, in examples of the present invention, in order to prove a function of BAT1 considered to be related to deactivation of the brassinosteroid hormone in addition to a function of the biomass increase and decrease, an amount of each intermediate metabolite of a brassinosteroid biosynthesis process of a BAT1-overexpressng transformant was measured through gas chromatography/mass spectrometry (GC-MS) (refer to FIG. 4). As expected, it was observed that brassinosteroid intermediate metabolites such as 6-deoxotyphasterol (6-deoxoTY), 6-deoxocastasterone (6-deoxoCS), or typhasterol (TY) statistically significantly decreased more than those of the wild type, in the BAT1-overexpressing transformant.

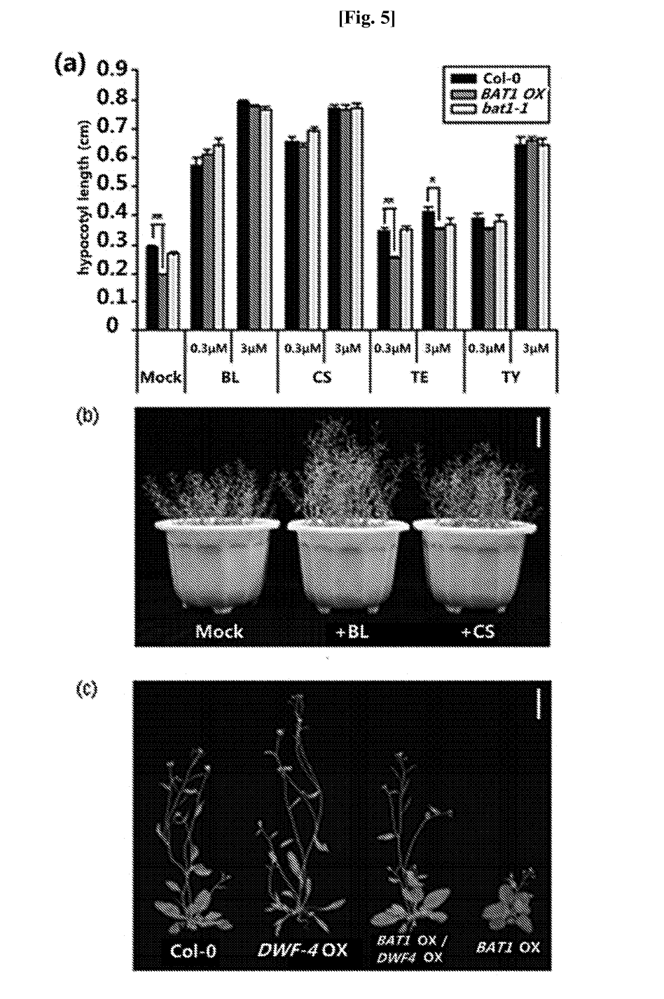

In addition, in examples of the present invention, in order to determine whether a phenotype related to deactivation of a brassinosteroid is restored by an external or internal brassinosteroid, a length of a hypocotyl, and phenotypes of a leaf and a stem of an adult were observed (refer to FIG. 5). It was observed that the BAT1 gene-overexpressing plant having a shorter hypocotyl height than that of the wild type has a length that significantly increased due to several brassinosteroid metabolites (BL; brassinolide, CS; castasterone, or TY; typhasterol) (refer to FIG. 5A). Also, it was observed that, when the BAT1 gene-overexpressing plant showing a dwarf phenotype even in an adult period was sprayed with the brassinosteroid metabolite, an overall stem length and a surface area of a small leaf increased (refer to FIG. 5B). Restoring of the phenotype of the BAT1 gene-overexpressing plant according to treatment with a brassinosteroid from the outside of the plant may be obtained by the internal brassinosteroid. It has been well known that a plant overexpressing DWF4 (DWARF4), which is an important gene of mediating biosynthesis of a brassinosteroid, has a significantly increased amount of a brassinosteroid and shows a phenotype in which lengths of a stem and a petiole increased ultimately, unlike the mutant defective in brassinosteroid signal transmission. It was observed that, when this DWF4 gene-overexpressing plant and the BAT1 gene-overexpressing plant whose biosynthesis of brassinosteroid is considered to be deactivated are genetically cross fertilized in the present invention, an excessively accumulated brassinosteroid in the DWF4 gene-overexpressing plant was deactivated due to BAT1 gene overexpression, and a phenotype similar to that of the wild type was shown ultimately (refer to FIG. 5C).

As described above, according to the present invention, it is obvious that both first and second growths of plants increased according to manipulation of the newly identified gene and the biomass increasing plants may be obtained accordingly. Therefore, it is expected that the BAT1 gene of the present invention and the transgenic plant bat1-1 thereof may be widely used in applications for biomass production.

Hereinafter, exemplary examples of the invention will be described for promoting an understanding of the invention. However, the following examples should be considered in a descriptive sense only and the scope of the invention is not limited to the following examples.

### Examples

### Example 1

### Selecting Mutant Having Increased Procambium Activity from Arabidopsis thaliana and Analyzing Phenotype of Vascular Tissues

### (1) Observation of Cross Section of Stem

Seeds of about 12,000 transgenic plants produced using a wild type of Arabidopsis thaliana (Col-0) and RAFL cDNA (RIKEN amplified Arabidopsis Full-Length cDNA) obtained from Rikagaku Kenkyusho (RIKEN). The plants were grown in a greenhouse whose temperature was regulated at about 23° C. under a long-day condition having a 24-hour period of 16-hour light condition/8-hour dark condition. Since the transgenic plants have different growth cycles despite having grown the same number of days, in order to observe the plants at the same stage, when one silique formed, the lowest part of the stem was cut to about 5 mm and obtained while being soaked in 3% glutaraldehyde in a 0.1 M sodium phosphate buffer solution (pH 7.2). The obtained sample was soaked in a solution, vacuumed for 10 minutes, reacted and fixed at 4° C. for 3 hours, and then general resin embedding was performed. While shaking the sample using a rocker at room temperature, the sample was washed with a 0.1 M sodium phosphate buffer solution (pH 7.2) for 20 minutes twice, and dehydrated while increasing 10% every 10 minutes from 30% to 100%. Acetone was changed to a second new 100% acetone, the sample was reacted for 8 to 9 hours, and then treated with a new 100% acetone again for 10 minutes. A resin was gradually increased in ratios to acetone of 1:3, 1:2, 1:1, and 2:1 at intervals of 1 hour for replacement, the 100% resin was replaced at intervals of 2 hours, and vacuumed for 10 minutes, and this process was repeated three times in total. The resin sample that was hardened in an oven at 65° C. for 24 to 48 hours using an embedded frame was cut to a fragment of 2 μm (using a glass knife or a diamond knife after the sample was trimmed to a rectangle or a trapezoid to show all tissues) and then stained with 0.05% toluidine blue to observe a cross section. Accordingly, a thickness of a cross-section of the stem of the plant in which the numbers of cells and layers of the procambium increased more than those of the wild type was measured using a microscope.

### (2) Identifying BAT1-Overexpressing Gene

In order to find a corresponding foreign gene showing various phenotypes in vascular tissues of the F23231 plant among about 12,000 FOX hunting systems, a genomic DNA of the F23231 plant was extracted. A leaf was quickly freezed using a liquid nitrogen, grounded using stainless steel beads, was added with an extraction solution (250 μl) containing 0.1 M Tris-Cl, 0.05 M EDTA 0.5 M NaCl, 1.25% SDS (pH 8.0), and treated at 65° C. for 15 minutes. A phenol:chloroform:isoamylalcohol (25:24:1) solution of the same volume (250 μl) was added and the solution was vertically shaken for 5 to 10 minutes. The solution was centrifuged at 4° C. and 13.000 rpm for 10 minutes, and a supernatant was transferred to a new tube. The solution was added with isopropanol of the same volume, vertically shaken 10 times and mixed, and then centrifuged at 13,000 rpm again for 1 minute. Then, a supernatant was removed, DNA pellets were washed with 70% ethanol and re-suspended in 30 CI water, and the genomic DNA was extracted. Primers (GS4, SEQ ID NO: 3, 5′-ACATTCTACAACTACATCTAGAGG-3′; GS6, SEQ ID NO: 4, 5′-CGGCCGCCCCGGGGATC-3′) specific to base sequences of a vector used in plant transformation were used to perform PCR on the genomic DNA. The amplified product was conducted with 1% agarose gel electrophoresis and stained with EtBr to determine a size. DNA was extracted from the gel, and cDNA base sequences of BAT1 (at4g31910) genes were determined by sequencing. In order to verify expression of this gene in the transgenic plant, qRT-PCR was performed using RNAs extracted from the stem.

### (3) Analyzing Expression of Marker Gene Specific to Brassinosteroid Signal Transmission

In addition to the change in the vascular tissues, in order to verify a phenotype related to signal transmission of a plant hormone brassinosteroid in a molecular level, RNA was extracted from a leaf of the plant using a Trizol reagent, and cDNA was synthesized using reverse transcription polymerase chain reaction (RT-PCR). qRT-PCR was performed using primers specific to brassinosteroid biosynthesis related genes, CPD, DWF4, and ROT3, and a control gene ACT2. These genes were used as marker genes that increased when there is no brassinosteroid signal.

### (4) Measurement of Length of Stem

The wild type, the BAT1-overexpressing transformant and the bat1-1-deficient mutant were grown in 0.5× B5 media for 7 days, and planted in the ground. After each stem was grown to a size of about 5 cm, a length of the stem was measured daily. As shown in FIGS. 2B and 2C, the BAT1-overexpressing transformant showed a dwarf phenotype in which a length of the stem significantly decreased more than that of the wild type. On the other hand, it was determined that the bat1-1-deficient mutant had a length of the stem that statistically significantly increased more than that of the wild type.

Cross sections of the stems observed by the above method were shown in FIG. 1A (the BAT1-overexpressing plant) and in FIG. 2D (the bat1-1-deficient mutant). In addition to contrasting the increase and decrease in the number of vascular bundles, a dwarf phenotype of the BAT1-overexpressing plant and a bat1-1 phenotype in which a length of the stem increased were shown in FIG. 1B, FIG. 1C, FIGS. 2B and 2C. Selection of a T-DNA insertion mutant and a schematic diagram thereof were shown in FIG. 2A. In the present invention, a bat1-1-deficient mutant inserted into a first exon was used. Also, as shown in FIG. 1D, it was determined that expression of genes known to function in biosynthesis of a plant hormone brassinosteroid significantly increased in the BAT1-overexpressing plant more than that of the wild type by the method.

### Example 2

### Identifying Temporal and Spatial Expression Pattern of BAT1 RNA

In order to observe an RNA expression pattern of BAT1, a promoter of the gene was recombined with a pCAMBIA 1303 (copyrighted by Cambia, Australia) binary vector. A primer in which EcoR I was inserted to 5′ and Nco I was inserted to 3′ specific to 2000 bp in front of a BAT1 gene promoter was used and amplification was performed by PCR. A plasmid pCAMBIAJ303-promoterBAT1-GUS was prepared such that the result was converged with the GUS protein and expressed. In order to express the result in the plant, agrobacterium was used for transformation, and transformation was performed on Arabidopsis thaliana using a floral dip method. A seed of the transformed plant was obtained. Independent plants growing in 0.5× MS, 30 mg/L hygromycin media were selected, planted in the ground, and grown. Seeds were separately obtained and planted again in media of the same composition. A part of a population in which a ratio of phenotypes of survivors and dead was 3:1 was planted in the ground, and a part thereof was stained to analyze the expression pattern. Expression of the GUS protein was observed such that the plant was soaked in 50 mM NaPO₄(pH7.0), 1 mM X0Gluc, 5 mM K₃Fe(CN)₆, 5 mM K₄Fe(CN)₆, 0.2% triton X-100 solution, reacted at 37° C. for 12 hours, a leaf, and a cotyledon and a root in a juvenile period were observed or various tissues of an adult were observed using a microscope.

As shown in FIG. 3, RNA encoded by BAT1 was expressed in vascular tissues of the leaf, the cotyledon and the root. Expression specific to vascular tissues of the stem could be observed in the adult. This result proved that a precondition that a phenotype shown in vascular tissues in the transgenic plant shows expression and functioning of BAT1 in vascular tissues was satisfied.

### Example 3

### Identifying BAT1 Protein Expression Pattern in Cell

In order to express the BAT1 protein in a protoplast of single mesophyll cell, a plasmid to be expressed in combination with a fluorescent protein GFP was recombined (35S promoter-BAT1-GFP). The recombinant plasmid was isolated at a high concentration of 2 μg/μl using CsCl and used. In order to obtain the protoplast of the single mesophyll cell from Arabidopsis thaliana, a wild type (Col-0) was grown for 3 to 4 weeks, and a leaf was finely cut, put into a 0.4 M mannitol, 20 mM KCl 20 mM MES, 1% cellulase, 0.25% macerozyme RIO, 10 mM CaCl₂, 0.1% BSA solution, vacuumed for 30 minutes while blocking light using a foil, and then reacted at room temperature for 3 to 4 hours. In general, BAT1 and marker genes expressed in each organelle were added to 2×10⁴ cells, 40 μg DNA was used as a transfectant, and expression in the cell was observed using a confocal microscope after 12 to 14 hours. FIG. 3A showed the results. As shown in FIG. 3A, it was determined that the BAT1 protein was positioned in a nucleus, and when BiP-RFP, a marker gene of an endoplasmic reticulum, was used as a transfectant together, was expressed at the same position precisely. Therefore, it is expected that a substrate in which the BAT1 protein serves as an acyltransferse enzyme is likely to be a protein in a nucleus, an endoplasmic reticulum or a cytoplasm or a secondary metabolite.

In conclusion, it can be understood that, since the BAT1 gene-deficient bat1-1 showed a phenotype in which biomass increased, this gene can be used for application crops and practicality can be increased.

### Example 4

### Measurement of Amount of Brassinosteroid of BAT1-Overexpressing Plant

A dwarf phenotype shown in the BAT1-overexpressing plant is similar to that of a mutant defective in brassinosteroid signal transmission. Therefore, when BAT1 genes are overexpressed, a change in an amount of a brassinosteroid in the plant was measured. As shown in FIG. 4, biosynthesis of a brassinosteroid was converted into castasterone (CS) or brassinolide (BL) having an activity through several intermediate metabolites. Research on biosynthetic pathways thereof, intermediate metabolites, or biosynthetic genes involved in conversion into each step was relatively well studied. However, research on deactivation of an active brassinosteroid, a storing form according to excessive synthesis and the like has not been well reported. In order to measure an amount of a brassinosteroid in the plant, the wild type (Col-0), the BAT1 gene-overexpressing plant and the bat1-1-deficient mutant each having a weight of 60 g and grown in a greenhouse under a long-day condition for 6 weeks were prepared and a series of experiments were conducted. The sample was frozen in order to preserve an original state and increase water solubility, and depressurized and left without change, thereby performing freezing and drying (lyophilization) of sublimating and removing water in the sample. The dried sample was prepared in a powder state using a mortar. 300 ml of 90% methanol was used to obtain an extracted solution three times, and a rotary evaporator was used to rotate a depressurized flask in a water tank containing hot water (45 to 50° C.) to concentrate the methanol solution. The fraction extract obtained by evaporation was partitioned into a solution three times using 500 ml of water and chloroform (CHCl₃) of 500 ml (partitioning). Then, the chloroform solution fraction was concentrated using the rotary evaporator as above, 200 ml of 80% methanol and 200 ml of n-hexane were used to partition the solution four times. The concentrated 80% methanol fraction extract was partitioned again three times using 300 ml of ethyl acetate and 300 ml of phosphate buffer (pH 7.8). The ethyl acetate fraction extract was concentrated by collecting activity fractions through silica gel column chromatography. As an elution solvent, 300 ml of chloroform solutions containing 1, 2, 3, 4, 5, 6, 7, 8, 9, 10, 20, 50, and 100% (v/v) methanol were used and dissolving and elution were performed through a column. 3% to 7% (v/v) methanol fractions were collected, depressurized, and concentrated, and purified using a SepPak C18 silica cartridge column. The obtained fraction in this manner was depressurized, concentrated, and dissolved in 30 ml of methanol. In order to obtain a purified product of a high purity, reverse phase high performance liquid chromatography (HPLC) (SenshuPak C18, 10×150 mm) was performed. 45% acetonitrile (MeCN) was flowed at a flow rate of 2.5 ml/min, a fraction was obtained for each minute, gas chromatograph/mass spectrophotometry (GC/MS) was used to compare and analyze amounts of each brassinosteroid intermediate metabolite of a control group and a detected value.

Therefore, it was observed that the BAT1 gene-overexpressing plant had a significantly decreased brassinosteroid more than that of the wild type, and particularly, decreased brassinosteroid intermediate metabolites such as 6-deoxotyphasterol (6-deoxoTY), 6-deoxocastasterone (6-deoxoCS), and TY (typhasterol).

### Example 5

### Functional Analysis of BAT1 Protein

Amino acid sequences of base sequences of an at4g31910/pGEM-T Easy vector containing cDNA of BAT1 were determined using In silico translation tool, BCM Search Launcher (http://searchlauncher.bcm.tmc.edu). The result showed that the amino acid sequences include 458 amino acids corresponding to the TAIR database, and 3 exons and 2 introns are included (refer to FIG. 2A). It can be seen that the BAT1 protein has a molecular weight of about 51.1 kDa, and has an isoelectric point (pI) value of 7.4946. When the BLAST database (http://www.ncbi.nlm.nih.gov/BLAST) was searched for amino acid sequences of BAT1, a high homology with genes including the acyltransferase domain was shown.

When a domain having a known function was searched for using an InterProScan program (http://www.ebi.ac.uk), it was determined that the acyltransferase domain was included in common with other acyltransferases. Therefore, it can be expected that a protein encoded by BAT1 has an acyltransferase activity.

### Example 6

### Phenotype Restoring of BAT1 Gene-Overexpressing Plant by Brassinosteroid

The wild type and the BAT1 gene-overexpressing plant were vertically grown in a greenhouse whose temperature was regulated at about 23° C. under a long-day condition having a 24-hour period of 16-hour light condition/8-hour dark condition in 0.5× B5 media. A hypocotyl of the vertical-grown plant was distinctively shown in the BAT1 gene-overexpressing plant. Various brassinosteroids were treated at various concentrations, and a height of the hypocotyl was measured after 7 days. As the brassinosteroid, brassinolide (BL), castasterone (CS), teasterone (TE), or typhasterol (TY) was added at a concentration of 0.3 μM or 3 μM to the medium.

In order to observe restoring of a phenotype of a stem length of an adult by a brassinosteroid, the BAT1 gene-overexpressing plant grown for 6 weeks under the long-day condition was sprayed with 1 μM of brassinolide (BL) and castasterone (CS), which are brassinosteroids known to have a high activity, once a week for 4 weeks. The restored phenotypes of the stem length decreased by each brassinosteroid were shown in pictures.

Biosynthesis of brassinosteroid is mediated by several known genes. Among them, a DWARF4 (DWF4) gene is the most important gene for the biosynthesis of a brassinosteroid. When the DWF4 gene is over-expressed, the plant shows a phenotype in which a height is great and a petiole is long and curved, which is shown in connection with a significantly increased amount of a brassinosteroid. In the present invention, in order to know an influence of BAT1 on a phenotype in which an amount of an internal brassinosteroid in the known DWF4 gene-overexpressing plant increased, two plants were genetically cross fertilized. A phenotype of a descendant obtained by cross fertilization of the two plants was shown in comparison with the BAT gene-overexpressing plant, the DWF4 gene-overexpressing plant, and the wild type.

The above description of the invention is only exemplary, and it will be understood by those skilled in the art that various manipulations can be made without departing from the scope of the present invention and without changing essential features. Therefore, the above-described examples should be considered in a descriptive sense only and not for purposes of limitation.

### INDUSTRIAL APPLICABILITY

The biomass production increasing gene of the present invention enables a transgenic plant having an increased cambium activity to be produced. Therefore, it is expected that the invention can contribute to supplement raw materials of pulp and paper industries and can be beneficially used to ensure raw materials of a bioethanol. Also, the invention can be expected to be beneficially used as heating and electricity production materials in the form of firewood, pellet or the like.

Sequence list Free Text

<210> 1
<211> 1377
<212> DNA
<213> Arabidopsis thaliana L. Heynh.
<400> 1
atgcccatgt taatggcgac acgtatcgat ataatccaaa
agcttaatgt atatccaagg tttcaaaacc atgacaagaa
gaaactaatc actctctcca atttggaccg tcagtgtcct
ttactcatgt actctgtctt cttctacaag aataccacaa
ctcgtgactt tgactccgtc ttctccaacc tgaagctcgg
gctggaggag actatgtctg tgtggtatcc cgcggcaggg
agactgggtt tggacggagg tggctgcaag ctcaacatcc
ggtgtaacga tggtggcgca gtcatggtgg aggcggtggc
gacaggtgtc aagttgtccg agcttggtga tttgactcag
tacaatgagt tttatgagaa tttagtttac aagccttcct
tggatggtga tttctctgtg atgcctcttg ttgttgctca
ggtgacaaga tttgcatgtg gaggttactc aattggaatc
ggtacaagcc actctctatt tgatggaatc tcagcttacg
aattcattca cgcgtgggcc tccaactctc acattcacaa
caaatccaac agcaagatta ctaataaaaa ggaagatgtg
gtcatcaaac cggttcatga tcgacgaaat ctactggtta
accgggatgc tgtccgagaa accaatgctg cagccatttg
tcatctgtac cagttgatca aacaggcgat gatgacctat
caggagcaaa accgtaactt agagttacca gactctggtt
ttgtgatcaa aacgttcgag cttaatggcg atgcgataga
aagcatgaag aagaaatcac tagaagggtt catgtgctcc
tcctttgagt ttcttgctgc tcatttgtgg aaggcaagaa
caagggcttt agggttgagg agagacgcca tggtgtgttt
acaattcgca gtggacataa ggaaaagaac ggagacaccg
ctgccagaag ggttttccgg caacgcatac gtgcttgcct
cggtggcatc caccgccaga gaattacttg aagagctaac
actcgagtca atagtcaaca agatcagaga agccaagaaa
tcaattgacc aaggttacat aaactcttac atggaagcac
tcggaggtag taatgacgga aatctccctc ctctcaaaga
gctaacccta atctccgact ggacaaaaat gccatttcac
aatgttggct ttggcaacgg cggcgagcca gcggattaca
tggccccact gtgtccaccg gtgccacaag ttgcttattt
catgaagaac cctaaagatg ccaaaggtgt tcttgtgagg
attggcttgg acccacgaga tgttaatggc ttttcaaatc
atttccttga ttgctaa
<210> 2
<211> 458
<212> PRT
<213> Arabidopsis thaliana L. Heynh.
<400> 2
Met Pro Met Leu Met Ala Thr Arg Ile Asp Ile
Ile Gln Lys Leu Asn Val Tyr Pro Arg Phe Gln
Asn His Asp Lys Lys Lys Leu Ile Thr Leu Ser
Asn Leu Asp Arg Gln Cys Pro Leu Leu Met Tyr
Ser Val Phe Phe Tyr Lys Asn Thr Thr Thr Arg
Asp Phe Asp Ser Val Phe Ser Asn Leu Lys Leu
Gly Leu Glu Glu Thr Met Ser Val Trp Tyr Pro
Ala Ala Gly Arg Leu Gly Leu Asp Gly Gly Gly
Cys Lys Leu Asn Ile Arg Cys Asn Asp Gly Gly
Ala Val Met Val Glu Ala Val Ala Thr Gly Val
Lys Leu Ser Glu Leu Gly Asp Leu Thr Gln Tyr
Asn Glu Phe Tyr Glu Asn Leu Val Tyr Lys Pro
Ser Leu Asp Gly Asp Phe Ser Val Met Pro Leu
Val Val Ala Gln Val Thr Arg Phe Ala Cys Gly
Gly Tyr Ser Ile Gly Ile Gly Thr Ser His Ser
Leu Phe Asp Gly Ile Ser Ala Tyr Glu Phe Ile
His Ala Trp Ala Ser Asn Ser His Ile His Asn
Lys Ser Asn Ser Lys Ile Thr Asn Lys Lys Glu
Asp Val Val Ile Lys Pro Val His Asp Arg Arg
Asn Leu Leu Val Asn Arg Asp Ala Val Arg Glu
Thr Asn Ala Ala Ala Ile Cys His Leu Tyr Gln
Leu Ile Lys Gln Ala Met Met Thr Tyr Gln Glu
Gln Asn Arg Asn Leu Glu Leu Pro Asp Ser Gly
Phe Val Ile Lys Thr Phe Glu Leu Asn Gly Asp
Ala Ile Glu Ser Met Lys Lys Lys Ser Leu Glu
Gly Phe Met Cys Ser Ser Phe Glu Phe Leu Ala
Ala His Leu Trp Lys Ala Arg Thr Arg Ala Leu
Gly Leu Arg Arg Asp Ala Met Val Cys Leu Gln
Phe Ala Val Asp Ile Arg Lys Arg Thr Glu Thr
Pro Leu Pro Glu Gly Phe Ser Gly Asn Ala Tyr
Val Leu Ala Ser Val Ala Ser Thr Ala Arg Glu
Leu Leu Glu Glu Leu Thr Leu Glu Ser Ile Val
Asn Lys Ile Arg Glu Ala Lys Lys Ser Ile Asp
Gln Gly Tyr Ile Asn Ser Tyr Met Glu Ala Leu
Gly Gly Ser Asn Asp Gly Asn Leu Pro Pro Leu
Lys Glu Leu Thr Leu Ile Ser Asp Trp Thr Lys
Met Pro Phe His Asn Val Gly Phe Gly Asn Gly
Gly Glu Pro Ala Asp Tyr Met Ala Pro Leu Cys
Pro Pro Val Pro Gln Val Ala Tyr Phe Met Lys
Asn Pro Lys Asp Ala Lys Gly Val Leu Val Arg
Ile Gly Leu Asp Pro Arg Asp Val Asn Gly Phe
Ser Asn His Phe Leu Asp Cys
<210> 3
<211> 24
<212> DNA
<213> Artificial Sequence
<220>
<223> GS4
<400> 3
acattctaca actacatcta gagg
<210> 4
<211> 17
<212> DNA
<213> Artificial Sequence
<220>
<223> GS6
<400> 4
cggccgcccc ggggatc

1. A composition of increasing biomass production of a plant comprising a base sequence encoding the amino acid sequence of SEQ ID NO: 2.

2. The composition of claim 1, wherein the base sequence comprises the base sequence of SEQ ID NO: 1.

3. A composition of increasing biomass production comprising a plant expression recombinant vector into which a base sequence encoding the amino acid sequence of SEQ ID NO: 2 is inserted.

4. A plant that is transformed using the composition according to claim 1, wherein the plant has increased biomass production.

5. A method of increasing biomass production of a plant, comprising transforming a plant using the composition according to claim 1.

6. A plant that is transformed using the composition according to claim 2, wherein the plant has increased biomass production.

7. A plant that is transformed using the composition according to claim 3, wherein the plant has increased biomass production.

8. A method of increasing biomass production of a plant, comprising transforming a plant using the composition according to claim 2.

9. A method of increasing biomass production of a plant, comprising transforming a plant using the composition according to claim 3.
